(19) United States
(12) Patent Application Publication
(10) Pub. No.: US 2007/0060860 A1
(43) Pub. Date: Mar. 15, 2007

(54) Iontophoresis device

(75) Inventors: Mizuo Nakayama, Shibuya-ku (JP); Kiyoshi Kanamura, Shibuya-ku (JP); Takehiko Matsumura, Shibuya-ku (JP); Hidero Akiyama, Shibuya-ku (JP); Akihiko Matsumura, Shibuya-ku (JP)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Shibuya-ku (JP)

(21) Appl. No.: 11/506,598

(22) Filed: Aug. 18, 2006

Related U.S. Application Data
(60) Provisional application No. 60/718,019, filed on Sep. 15, 2005.

(30) Foreign Application Priority Data
Aug. 18, 2005 (JP) 2005-238026

(51) Int. Cl. A61N 1/30

(52) U.S. Cl. 604/20

(57) ABSTRACT

One or more electrodes of an iontophoresis device may include a composite ion exchange membrane comprising a first ion exchange membrane of a first polarity and a second ion exchange membrane of a second polarity, or a first ion exchange membrane of the first polarity, a semi-permeable membrane, and a second ion exchange membrane of the second polarity. The respective membranes may be integrally coupled together. This may lead to simplified production processes, automated production, mass production, and reductions in production costs for the iontophoresis device.

### CROSS-REFERENCE TO RELATED APPLICATION

This application claims the benefit under 35 U.S.C. § 119(e) of U.S. Provisional Patent Application No. 60/718,019, filed Sep. 15, 2005, and Japan Patent Application No. 2005-238026, filed Aug. 18, 2005, where these two applications are incorporated herein by reference in their entireties.

### BACKGROUND OF THE INVENTION

1. Field

The present disclosure relates to an iontophoresis device that administers active agent ions to a subject by driving the active agent ions with an electric potential having the same polarity as that of the active agent ions.

2. Description

An iontophoresis device generally includes an active electrode assembly holding active agent ions that dissociate into positive or negative ions, and a counter electrode assembly that functions as a counter electrode to the active electrode assembly. The active agent ions are administered to a subject by the application of an electric potential having the same polarity as that of the active agent ions to the active electrode assembly, under the condition that both assemblies are in contact with the biological interface of the subject.

WO 03/037425 discloses an iontophoresis device that has a high active administration agent efficiency, and which may be capable of preventing the decomposition of the active agent at the time of energization.

FIG. 5 is an explanatory view that shows the iontophoresis device disclosed in WO 03/037425.

The iontophoresis device of WO 03/037425 comprises: an active electrode assembly 110 comprising an electrode 111, to which an electric potential of a first polarity is applied from an electric power source 130, an electrolyte solution reservoir 112 that holds an electrolyte solution; an ion exchange membrane 113 of a second polarity, an active agent solution reservoir 114 that holds an active agent solution containing active agent ions of the first polarity, and an ion exchange membrane 115 of the first polarity; and a counter electrode assembly 120 comprising: an electrode 121 to which an electric potential of the second polarity is applied from the electric power source 130, an electrolyte solution reservoir 122 that holds an electrolyte solution, an ion exchange membrane 123 of the first polarity, an electrolyte solution reservoir 124 that holds an electrolyte solution, and an ion exchange membrane 125 of the second polarity.

The transfer of ions present on the surface of, or inside, a subject and have a polarity opposite to that of the active agent ions (hereinafter referred to as “biological counter ions”) to the active agent solution reservoir 114 may be blocked because the ion exchange membrane 115 interposes between the active agent solution reservoir 114 and a biological interface such as skin. Therefore, the amount of a current consumed by the movement of the biological counter ions decreases, and the administration efficiency of the active agent ions increases. In addition, the decomposition of an active agent near the electrode 111 upon energization may be prevented because the transfer of the active agent ions to the electrolyte solution reservoir 112 may be blocked by the ion exchange membrane 113.

The applicant has proposed an iontophoresis device made by improving the active electrode assembly 110 in the iontophoresis device of WO 03/037425, and has filed this as U.S. Patent Provisional Application 60/693,668 (hereinafter referred to as the '668 application.)

FIG. 6A is an explanatory view showing an active electrode assembly 210 disclosed as an embodiment in the '668 application.

The active electrode assembly 210 comprises an electrode 211 to which an electric potential of the first polarity is applied, an electrolyte solution reservoir 212 that holds an electrolyte solution, an ion exchange membrane 213 of the second polarity, and an ion exchange membrane 215 of the first polarity, the ion exchange membrane 215 being doped with active agent ions of the first polarity.

An iontophoresis device including the active electrode assembly 210 achieves effects similar to those of the iontophoresis device of WO 03/037425. For example, the efficiency of the administration of an active agent may increase because the transfer of a biological counter ion may be blocked by the ion exchange membrane 215. In addition, the decomposition of the active agent at the time of energization may be prevented because the transfer of the active agent ions to the electrolyte solution reservoir 212 may be blocked by the ion exchange membrane 213.

In addition, the iontophoresis device including the active electrode assembly 210 may achieve additional effects. For example, the efficiency of the administration of the active agent may increase further because the active agent ions are held by the ion exchange membrane 215, which is provided in close proximity to the biological interface of a subject. In addition, the stability and preservability of the active agent ions may increase because the active agent ions are held bound to exchange groups in the ion exchange membrane 215. The active agent solution reservoir 114, which must be handled in a wet state, can thus be omitted from the assembly of the active electrode assembly 210.

The applicant has proposed another improved iontophoresis device, and has filed this as JP 2005-222893 A (hereinafter referred to the '893 application.)

FIGS. 6B and 6C are explanatory views showing an active electrode assembly 310 and a counter electrode assembly 320 disclosed as an embodiment of the '893 application.

The active electrode assembly 310 comprises an electrode 311 to which an electric potential of the first polarity is applied, an electrolyte solution reservoir 312 that holds an electrolyte solution, an ion exchange membrane 313 of the first polarity, an ion exchange membrane 313′ of the second polarity, an active agent solution reservoir 314 that holds an active agent solution containing active agent ions of the first polarity, and an ion exchange membrane 315 of the first polarity. The counter electrode assembly 320 comprises an electrode 321 to which an electric potential of the second polarity is applied, an electrolyte solution reservoir 322 that holds an electrolyte solution, an ion exchange membrane 323 of the second polarity, an ion exchange membrane 323′ of the first polarity, an electrolyte solution reservoir 324 that holds an electrolyte solution, and an ion exchange membrane 325 of the second polarity.

An iontophoresis device including the active electrode assembly 310 achieves effects similar to those of the iontophoresis device of WO 03/037425. For example, the efficiency of the administration of an active agent may increase and decomposition of the active agent at the time of energization may be prevented due to the presence of the ion exchange membranes 313′ and 315.

In addition, in the active electrode assembly 310, the two ion exchange membranes 313 and 313′ having opposite polarities are arranged between the electrolyte solution reservoir 312 and the active agent solution reservoir 314, so transfer of ions between the electrolyte solution reservoir 312 and the active agent solution reservoir 314 during the storage of the device can be blocked. Therefore, an additional effect, that is, the prevention of the alteration of an active agent during the storage of the device resulting from the transfer of ions of the second polarity in the electrolyte solution reservoir 312 to the active agent solution reservoir 314 is achieved.

In an iontophoresis device including the counter electrode assembly 320, the two ion exchange membranes 323 and 323′ having opposite polarities are arranged between the electrolyte solution reservoir 322 and the electrolyte solution reservoir 324. The transfer of ions between the two electrolyte solution reservoirs 322 and 324 during the storage of the device may thus be blocked. Electrolytes having different compositions may be used for the electrolyte solution reservoirs 322 and 324. For example, an electrolyte solution suited to preventing and buffering an electrode reaction may be used in the electrolyte solution reservoir 322, and an electrolyte solution more suitable for subject contact may be used for the electrolyte solution reservoir 324. Mixing of the electrolyte solutions of both the electrolyte solution reservoirs during storage of the device may be prevented by the two ion exchange membranes 323 and 323′.

### BRIEF SUMMARY OF THE INVENTION

In one aspect, the present disclosure is directed to an iontophoresis device comprising an electrode assembly that includes a composite ion exchange membrane. The composite ion exchange membrane comprises a first ion exchange membrane of a first polarity and a second ion exchange membrane of a second polarity arranged on and integrally coupled to the first ion exchange membrane.

In one aspect, the present disclosure is directed to a composite ion exchange membrane. The composite ion exchange membrane comprises a first ion exchange membrane of a first polarity and a second ion exchange membrane of a second polarity arranged on and integrally coupled to the first ion exchange membrane.

The composite ion exchange membrane may be used in the active electrode assembly of the iontophoresis device disclosed in the '668 application, such as the ion exchange membrane 213 and the ion exchange membrane 215 of the first polarity 215 in the active electrode assembly 210. Similarly, the composite ion exchange membrane may be used in the active electrode assembly or counter electrode assembly of the iontophoresis device disclosed in the '893 application, such as the ion exchange membrane 313 and the ion exchange membrane 313′ in the active electrode assembly 310, or the ion exchange membrane 323 and the ion exchange membrane 323′ in the counter electrode assembly 320.

As a result of the first ion exchange membrane and the second ion exchange membrane being integrally coupled, production of the composite ion exchange membrane may be simplified, leading to automated production, mass production, and reduced production costs.

The first ion exchange membrane and the second ion exchange membrane may be integrally coupled by using a variety of methods, including: superimposing the first and second ion exchange membranes on each other and subjecting the resultant to thermocompression bonding; joining the first and second ion exchange membranes together using an adhesive; and applying an ion exchange resin to the first or second ion exchange membrane and curing the applied ion exchange resin to form the second or first ion exchange membrane.

The first and second ion exchange membranes should be coupled together having sufficient adhesion to not easily separate during handling and electrode assembly production.

In one aspect, the present disclosure is directed to an electrode assembly comprising a composite ion exchange membrane comprising of a first ion exchange membrane of the first polarity, a semi-permeable membrane laminated on the first ion exchange membrane, and a second ion exchange membrane of the second polarity laminated on the semi-permeable membrane, where the first ion exchange membrane, the semi-permeable membrane, and the second ion exchange membrane are integrally coupled together.

In one aspect, the present disclosure is directed to a composite ion exchange membrane for iontophoresis comprising of a first ion exchange membrane of the first polarity, a semi-permeable membrane laminated on the first ion exchange membrane, and a second ion exchange membrane of the second polarity laminated on the semi-permeable membrane, where the first ion exchange membrane, the semi-permeable membrane, and the second ion exchange membrane are integrally coupled together.

The composite ion exchange membrane may be used in the active electrode assembly of the iontophoresis device disclosed in the '668 application or in the active electrode assembly or counter electrode assembly of the iontophoresis device disclosed in the '893 application.

As a result of the first ion exchange membrane, the semi-permeable membrane, and the second ion exchange membrane being integrally coupled, production of the composite ion exchange membrane may be simplified, leading to automated production, mass production, and reduced production costs.

The first ion exchange membrane and the second ion exchange membrane may be integrally coupled by using a variety of methods. Such methods may include: superimposing those three membranes on one another and subjecting the resultant to thermocompression bonding; using an adhesive present at an interface between the first ion exchange membrane and the semi-permeable membrane, and an adhesive present at an interface between the semi-permeable membrane and the second ion exchange membrane; and applying an ion exchange resin to each of both surfaces of the semi-permeable membrane and curing the applied ion exchange resin to form each of the first and second ion exchange membranes.

The first ion exchange membrane, the semi-permeable membrane, and the second ion exchange membrane should be coupled together having sufficient adhesion to not easily separate during handling and electrode assembly production.

An active agent to be administered to a subject may be held by each of two electrode assemblies connected to both poles of an electric power source (each of the electrode assemblies serving both as an active electrode assembly and a counter electrode assembly). In addition, iontophoresis devices having multiple electrode assemblies connected to respective poles of the electric power source may also be employed. The composite ion exchange membranes described above may be used in one or more of the electrode assemblies in any of the iontophoresis devices.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is an explanatory view showing an iontophoresis device.

FIGS. 2A and 2B are explanatory sectional views each showing an active electrode assembly of the iontophoresis.

FIGS. 3A to 3F are explanatory sectional views each showing an iontophoresis device.

FIGS. 4A to 4D are explanatory sectional views each showing the a counter electrode assembly of an iontophoresis device.

FIG. 5 is an explanatory view showing a conventional iontophoresis device.

FIGS. 6A to 6C are explanatory views showing an active electrode assembly and a counter electrode assembly of an iontophoresis device described in another patent application by the present applicant.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices, controllers, electric potential or current sources and/or membranes have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “another embodiment” means that a particular referent feature, structure, or characteristic described in connection with the embodiment is included in at least one embodiment. Thus, the appearances of the phrases “in one embodiment,” or “in an embodiment,” or “another embodiment” in various places throughout this specification are not necessarily all referring to the same embodiment. Further more, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to a system for evaluating an iontophoretic active agent delivery including “a controller ” includes a single controller, or two or more controllers. It should also be noted that the term “or” is generally employed in its sense including “and/or” unless the content clearly dictates otherwise.

As used herein the term “membrane” means a boundary, a layer, barrier, or material, which may, or may not be permeable. The term “membrane” may further refer to an interface. Unless specified otherwise, membranes may take the form a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein, the term bipolar membrane means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules a first rate, and some other molecules a second rate different than the first. In yet further embodiments, the “semi-permeable membrane” may take the form of a selectively permeable membrane allowing only certain selective molecules to pass through it.

As used herein, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles.) In some embodiment a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

As used herein, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

As used herein, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., an active agent, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent. In some embodiments, the term “active agent” further refers to the active agent, as well as its pharmacologically active salts, pharmaceutically acceptable salts, proactive agents, metabolites, analogs, and the like. In some further embodiment, the active agent includes at least one ionic, cationic, ionizeable and/or neutral therapeutic active agent and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. While other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH. The term “active agent” may also refer to neutral agents, molecules, or compounds capable of being delivered via electro-osmotic flow. The neutral agents are typically carried by the flow of, for example, a solvent during electrophoresis. Selection of the suitable active agents is therefore within the knowledge of one skilled in the art.

Non-limiting examples of such active agents include lidocaine, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCI, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic active agents; zolpidem tartrate and similar sleep inducing agents; L-dopa and other anti-Parkinson's medications; aripiprazole, olanzapine, quetiapine, risperidone, clozapine and ziprasidone as well as other neuroleptica; diabetes active agents such as exenatide; as well as peptides and proteins for treatment of obesity and other maladies.

As used herein, the term “subject” generally refers to any host, animal, vertebrate, or invertebrate, and includes fish, mammals, amphibians, reptiles, birds, and particularly humans.

As used herein, the term “biological interface” refers to a surface of a subject to which an active agent can be administered by iontophoresis, and includes mucosa and skin.

As used herein, the term “transport number” refers to a ratio of a charge amount conveyed by the passage of an active agent counter ion through the second ion exchange membrane to the total charge conveyed through the second ion exchange membrane when an electrical potential of the first polarity is applied to the side of an electrolyte solution held by the electrolyte solution reservoir when the second ion exchange membrane is placed between the electrolyte solution and an active agent solution containing appropriate concentrations of active agent ions and active agent counter ion (for example, an active agent solution used for doping a first ion exchange membrane with active agent ions).

The headings provided herein are for convenience only and do not interpret the scope or meaning of the embodiments.

FIG. 1 is an explanatory view showing an iontophoresis device X.

An iontophoresis device for administering an active agent whose active agent component dissociates to cationic active agent ions (for example, lidocaine hydrochloride or morphine hydrochloride) will be exemplified herein, for convenience. An iontophoresis device for administering an active agent whose active agent component dissociates to anionic active agent ions (for example, ascorbic acid) may be made by reversing the poles of an electric power source, the polarity of each ion exchange membrane, and the polarity of ions with which a doping layer or a cation exchange membrane may be doped, compared to those described below for a cationic active agent.

The iontophoresis device X comprises: an electric power source 30; an active electrode assembly 10 coupled to the positive pole of the electric power source 30 through an supply line 31; and a counter electrode assembly 20 coupled to the negative pole of the electric power source 30 through an supply line 32.

A space capable of accommodating various assemblies described below is formed in the active electrode assembly 10 and the counter electrode assembly 20. The active electrode assembly 10 includes a container 16 with an open lower portion 16b. The counter electrode assembly 20 includes a container 26 with an open lower portion 26b. The containers 16 and 26 may be formed by using a variety of materials such as a plastic. The containers 16 and 26 may be formed by using a flexible material, for example, which may help to prevent the evaporation of water from the inside and the mixing in of foreign matter from the outside, and may also allow the iontophoresis device X follow the movement of a subject and/or irregularities in a biological interface on which the iontophoresis device X is placed. In addition, a removable liner composed of an appropriate material for preventing the evaporation of water or the mixing in of foreign matter during storage of the iontophoresis device X may be disposed on the lower portion 16b of the container 16 and on the lower portion 26b of the container 26. An adhesive layer for improving adhesiveness with the biological interface upon administration of an active agent can be disposed on the lower end 16e of the container 16 and on the lower end 26e of the container 26.

A battery, a constant electric potential source, a constant current source, a constant electric potential / current device, or the like may be used as the electric power source 30.

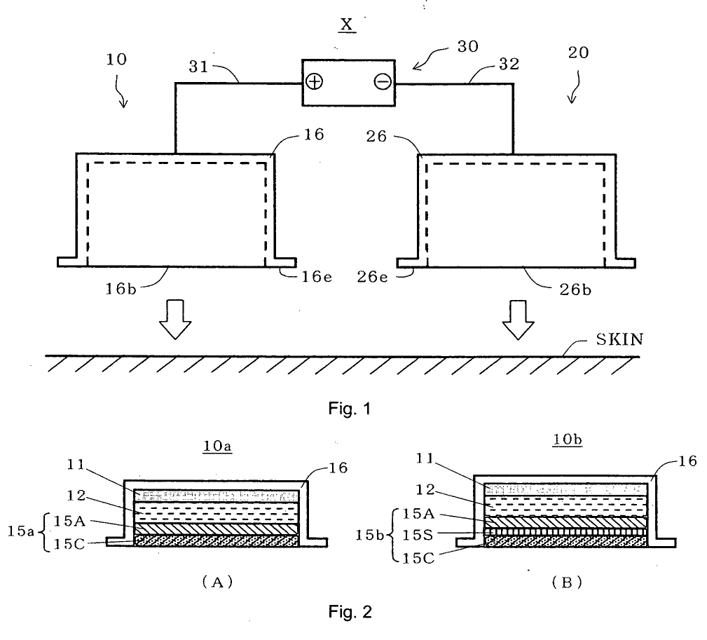

FIGS. 2A and 2B are explanatory sectional views showing active electrode assemblies 10a and 10b, each of which may be used as the active electrode assembly 10 of the iontophoresis device X.

The active electrode assembly 1Oa comprises: the electrode 11 connected to the supply line 31 of the electric power source 30; the electrolyte solution reservoir 12 that holds an electrolyte solution in contact with the electrode 11; and a composite ion exchange membrane 1 5a arranged on the front surface side of the electrolyte solution reservoir 12.

The composite ion exchange membrane 15a comprises an anion exchange membrane 15A arranged in contact with the electrolyte solution of the electrolyte solution reservoir 12 and a cation exchange membrane 15C arranged on the front surface side of the anion exchange 15A and doped with positive active agent ions. The anion exchange membrane 15A and the cation exchange membrane 15C are coupled together.

The anion exchange membrane 15A and the cation exchange membrane 15C may be coupled together by using a variety of methods. Examples of such methods include: joining the anion exchange membrane 15A and the cation exchange membrane 15C through thermocompression bonding; applying a cation exchange resin to the anion exchange membrane 15A and curing the applied cation exchange resin to form the cation exchange membrane 15C; an anion exchange resin to the cation exchange membrane 15C and curing the applied anion exchange resin to form the anion exchange membrane 15A; and applying an adhesive between the anion exchange membrane 15A and the cation exchange membrane 15C and joining them with each other by means of the adhesive.

The cation exchange membrane 15C may be doped with active agent ions by being immersed in an active agent solution containing the active agent ions. The amount of active agent ions with which the cation exchange membrane 15C is doped can be controlled based on the concentration of active agent ions in the active agent solution, immersion time, and the number immersions performed. The cation exchange membrane 15C may be doped with active agent ions before or after being coupled with the anion exchange membrane 15A.

Positive ions in the electrolyte solution reservoir 12 should be able to pass through the anion exchange membrane 15A in the active electrode assembly 10a when a positive electric potential is applied to the electrode 11. Therefore, an anion exchange membrane having a relatively low transport number, for example, 0.7 to 0.98, may be used.

The transport number of the anion exchange membrane 15A is defined as a ratio of the amount of charge conveyed by the passing of a negative ion in an active agent solution containing a suitable concentration of active agent ions (for example, an active agent solution used for doping the cation exchange membrane 15C with active agent ions) through the anion exchange membrane 15A to the total charge conveyed via the anion exchange membrane 15A when an electric potential of the first polarity is applied to the side of the electrolyte solution of the electrolyte solution reservoir 12 in a state where the anion exchange membrane 15A is arranged between the electrolyte solution and the active agent solution.

Similarly, when an adhesive is used for joining the anion exchange membrane 15A and the cation exchange membrane 15C with each other, the adhesive should allow positive ions in the electrolyte solution of the electrolyte solution reservoir 12 to pass.

The electrolyte solution reservoir 12 may hold an electrolyte solution into which an arbitrary electrolyte is dissolved. When an electrolyte having an oxidation potential lower than that of the electrolysis of water is used or a buffer electrolyte solution into which a plurality of electrolytes are dissolved is used, the generation of an oxygen gas or a hydrogen ions upon energization may be reduced, and changes in pH due to the generation of hydrogen ions may also be reduced.

If the mobility of positive ions in the electrolyte solution reservoir 12 is larger than that of active agent ions with which the cation exchange membrane 15C is doped, the positive ions may preferentially transfer to a subject more quickly than the active agent ions do, thus reducing the efficiency of the administration of the active agent ions. By keeping positive ions having a mobility larger than that of the active agent ions out of the reservoir, reductions in efficiency may be reduced.

The electrolyte solution reservoir 12 may hold an electrolyte solution in a liquid state. Alternatively, the electrolyte solution reservoir 12 may hold an electrolyte solution impregnated on an appropriate absorbing carrier such as gauze, filter paper, or an aqueous gel.

The iontophoresis device X including the active electrode assembly 10a administers active agent ions via a mechanism similar to that of the iontophoresis device disclosed in the '668 application.

That is, a positive electric potential may be applied to the electrode 11 with the cation exchange membrane 15C brought into contact with the biological interface of a subject. The active agent ions doped in the cation exchange membrane 15C may then transfer to the subject. Without being limited by theory, Applicants believe that positive ions in the electrolyte solution reservoir 12 transfer to the cation exchange membrane 15C via the anion exchange membrane 15A to replace the active agent ions that have transferred to the subject.

The composite ion exchange membrane 15a obtained by integrating the anion exchange membrane 15A and the cation exchange membrane 15C may be used in the iontophoresis device X. Assembly of the active electrode assembly 10a may thus be simplified, automated production and mass production may become easier, and production costs may be reduced.

In the active electrode assembly 10a, the electrolysis of water may occur upon energization between the anion exchange membrane 15A and the cation exchange membrane 15C, causing a reduction in efficiency of active agent administration and causing fluctuations in pH at a biological interface. Energization conditions, and/or the transport numbers of the anion exchange membrane 15A and/or the cation exchange membrane 15C, may therefore be adjusted so that the electrolysis of water does not occur, or, even if it occurs, the extent of the electrolysis will fall within an allowable range.

An active electrode assembly 10b is similar to the active electrode assembly 10a except that it includes a composite ion exchange membrane 15b instead of the composite ion exchange membrane 15a.

The composite ion exchange membrane 15b comprises the anion exchange membrane 15A, a semi-permeable membrane 15S arranged on the front surface side of the anion exchange membrane 15A, and the cation exchange membrane 15C arranged on the front surface side of the semi-permeable membrane 15S and doped with active agent ions. The anion exchange membrane 15A, the semi-permeable membrane 15S, and the cation exchange membrane 15C are integrally coupled.

Coupling may be performed by using a method similar to those described above for the composite ion exchange membrane 15a, such as joining through thermocompression bonding; forming the anion exchange membrane 15A and/or the cation exchange membrane 15C on the semi-permeable membrane 15S; or using an adhesive.

The cation exchange membrane 15C may be doped with active agent ions by means similar to those described above for the composite ion exchange membrane 15a.

An arbitrary semi-permeable membrane that allows passage of a positive ion in the electrolyte solution of the electrolyte solution reservoir 12 may be used for the semi-permeable membrane 15S. For example, an aqueous gel matrix such as an acrylic aqueous gel or a polyurethane based aqueous gel, or a membrane filter such as filter paper or a molecular cutoff membrane, may be used.

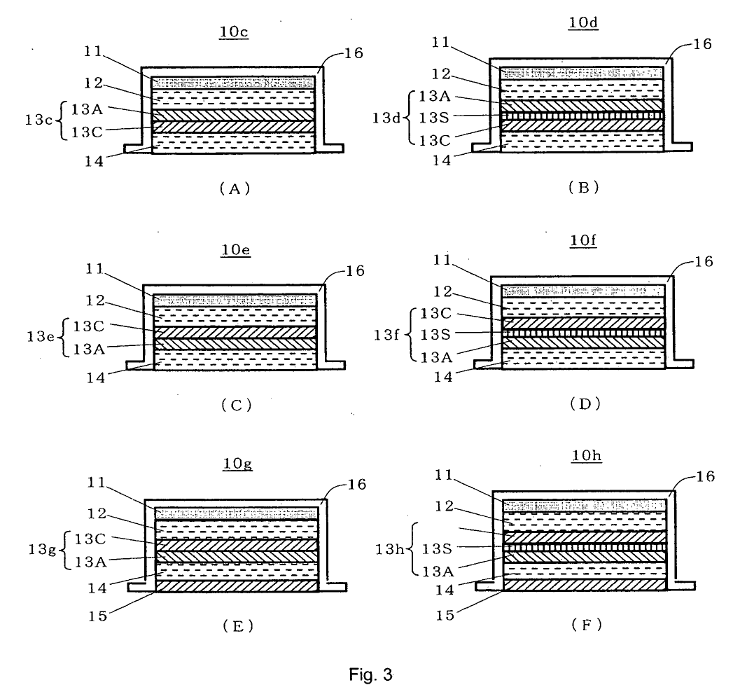

FIGS. 3A to 3F are explanatory sectional views showing active electrode assemblies 10c to 10h, each of which may be used as the active electrode assembly 10 of the iontophoresis device X.

The active electrode assembly 10c comprises: the electrode 11 connected to the supply line 31 of the electric power source 30; the electrolyte solution reservoir 12 that holds an electrolyte solution in contact with the electrode 11; a composite ion exchange membrane 13c arranged on the front surface side of the electrolyte solution reservoir 12; and the active agent solution reservoir 14 that holds an active agent solution, the active agent solution reservoir 14 being arranged on the front surface side of the composite ion exchange membrane 13c.

The composite ion exchange membrane 13c comprises an anion exchange membrane 13A arranged in contact with the electrolyte solution of the electrolyte solution reservoir 12 and a cation exchange membrane 13C arranged in contact with the active agent solution of the active agent solution reservoir 14. The anion exchange membrane 13A and the cation exchange membrane 13C are integrally coupled together in a manner similar to that used with the composite ion exchange membrane 15a.

The composite ion exchange membrane 13c should allow passage of positive ions in the electrolyte solution reservoir 12 and/or negative ions in the active agent solution reservoir 14 when the iontophoresis device X is energized. Therefore, an ion exchange membrane having a relatively low transport number, for example 0.7 to 0.98, may used for the anion exchange membrane 13A and/or the cation exchange membrane 13C.

The transport number of the anion exchange membrane 13A is defined as a ratio of the amount of charge conveyed by the passing of a negative ion in the active agent solution of the active agent solution reservoir 14 through the anion exchange membrane 13A to the total charge conveyed via the anion exchange membrane 13A when a positive electric potential is applied to the side of the electrolyte solution of the electrolyte solution reservoir 12 in a state where the anion exchange membrane 13A is arranged between the electrolyte solution and the active agent solution of the active agent solution reservoir 14. The transport number of the cation exchange membrane 13C is defined as a ratio of the amount of charge conveyed by the passing of a positive ion in the electrolyte solution of the electrolyte solution reservoir 12 through the cation exchange membrane 13C to the total charge conveyed via the cation exchange membrane 13C when a positive electric potential is applied to the side of the electrolyte solution in a state where the cation exchange membrane 13C is arranged between the electrolyte solution and the active agent solution of the active agent solution reservoir 14.

The electrolyte solution reservoir 12 can hold an electrolyte solution into which an arbitrary electrolyte is dissolved. When an electrolyte having an oxidation potential lower than that required for the electrolysis of water is used, or a buffer electrolyte solution into which multiple kinds of electrolytes are dissolved is used, the generation of oxygen gas or hydrogen ion upon energization may be reduced, and changes in pH due to the generation of hydrogen ions may be reduced.

The active agent solution reservoir 14 holds a solution of an active agent whose active agent component dissociates into positive active agent ions. The active agent solution reservoir 14 may hold the active agent solution in a liquid state. Alternatively, the active agent solution reservoir 14 may hold the active agent solution impregnated in a suitable appropriate absorbing carrier such as gauze, filter paper, or an aqueous gel.

The two ion exchange membranes 13A and 13C having opposite polarities are arranged between the electrolyte solution reservoir 12 and the active agent solution reservoir 14, so the transfer of active agent ions in the active agent solution reservoir 14 to the electrolyte solution reservoir 12 and the transfer of negative ions in the electrolyte solution reservoir 12 to the active agent solution reservoir 14 during the storage of the device may be blocked. Decomposition of the active agent near the electrode 11 upon energization may thus be prevented, and changes to the active agent in the active agent solution reservoir 14 during storage of the device may be prevented.

The transfer of the active agent ions or the negative ion in the electrolyte solution reservoir 12 during the storage of the device may be substantially suppressed even if the anion exchange membrane 13A or cation exchange membrane 13C has a relatively low transport number (a transport number of 0.7 to 0.98), as described above.

Furthermore, the composite ion exchange membrane 13a obtained by integrally coupling the anion exchange membrane 13A and the cation exchange membrane 13C may be used in the iontophoresis device X including the active electrode assembly 10c. Assembly of the active electrode assembly 10c may thus be simplified, automated and mass production may be simplified, and reductions in production costs may be achieved.

The electrolysis of water may occur between the anion exchange membrane 13A and the cation exchange membrane 13C in the active electrode assembly 10c. This may cause a reduction in efficiency of the administration of an active agent and a fluctuation in pH at a biological interface. Energization conditions, and/or the transport numbers of the anion exchange membrane 13A and the cation exchange membrane 13C may be adjusted so that the electrolysis of water does not occur, or, even if the electrolysis occurs, the extent of the electrolysis falls within an allowable range.

An active electrode assembly 10d is similar to the active electrode assembly 10c except that it includes a composite ion exchange membrane 13d instead of the composite ion exchange membrane 13c.

The composite ion exchange membrane 13d comprises the anion exchange membrane 13A, a semi-permeable membrane 13S arranged on the front surface side of the anion exchange membrane 13A, and the cation exchange membrane 13C arranged on the front surface side of the semi-permeable membrane 13S. The anion exchange membrane 13A, the semi-permeable membrane 13S, and the cation exchange membrane 13C are integral.

Coupling may be performed using a method similar to that used for the composite ion exchange membrane 15a.

The anion exchange 13A and cation exchange membrane 13C of the composite ion exchange membrane 13c may be used for the anion exchange membrane 13A and cation exchange membrane 13C of the composite ion exchange membrane 13d.

Any of a variety of membranes may be used for the semi-permeable membrane 13S as long as the membrane allows positive ions in the electrolyte solution of the electrolyte solution reservoir 12 to pass. For example, an aqueous gel matrix such as an acrylic aqueous gel or a polyurethane-based aqueous gel, or a membrane filter such as filter paper or a molecular cutoff membrane, may be used.

The active electrode assembly 10d may be used in a manner similar to that described above for the active electrode assembly 10c, and may achieve effects similar to those of the active electrode assembly 10c. Furthermore, the active electrode assembly 10d may prevent or reduce the occurrence of water electrolysis between the anion exchange membrane 13A and the cation exchange membrane 13C because the anion exchange membrane 13A and the cation exchange membrane 13C are separated from each other by the semi-permeable membrane 13S.

The active electrode assembly 10e is similar to the active electrode assembly 10c except that the orientation of a composite ion exchange membrane 13e is opposite to that of the active electrode assembly 10c. The active electrode assembly 10f is similar to the active electrode assembly 10d except that the orientation of a composite ion exchange membrane 13f is opposite to that of the active electrode assembly 10d.

That is, in the active electrode assemblies 10e and 10f, the cation exchange membrane 13C is arranged in contact with the electrolyte solution of the electrolyte solution reservoir 12, and the anion exchange membrane 13A is arranged in contact with the active agent solution of the active agent solution reservoir 14.

The active electrode assemblies 10e and 10f may make it more difficult for the electrolysis of water to occur between the anion exchange membrane 13A and the cation exchange membrane 13C compared to the active electrode assemblies 10c and 10d. This is because an ion exchange membrane having the same polarity as that of an electric potential (positive) to be applied to the electrode 11 (the cation exchange membrane 13C) is provided on a side proximate to the electrode 11 and an ion exchange membrane opposite in polarity to the electric potential (the anion exchange membrane 13A) is provided on a side distal from the electrode 11.

The active electrode assembly 10g is similar to the active electrode assembly 10e, further comprising a cation exchange membrane 15 on the front surface side of the active agent solution reservoir 14. The active electrode assembly 10h is similar to the active electrode assembly 10f, further comprising a cation exchange membrane 15 on the front surface side of the active agent solution reservoir 14

The iontophoresis device X including the active electrode assembly 10g or the active electrode assembly 10h may be used to administer active agent ions to a subject by applying a positive electric potential to the electrode 11 when the cation exchange membrane 15 contacts the biological interface of the subject.

The iontophoresis device X including the active electrode assembly 10g or the active electrode assembly 10h may increase the efficiency of active agent administration because the cation exchange membrane 15 may block the transfer of a biological counter ion to the active agent solution reservoir 14.

An active electrode assembly (not shown) obtained by placing a cation exchange membrane on the front surface side of the active agent solution reservoir 14 of each of the active electrode assemblies 10c and 10d (the active electrode assembly is referred to as an active electrode assembly 10i or an active electrode assembly 10j, respectively) may also increase active agent administration efficiency.

In each of the active electrode assemblies 10c, 10e, and 10g, the anion exchange membrane 13A or the cation exchange membrane 13C may have a molecular weight cut-off, thereby substantially blocking passage of electrolyte molecules in the electrolyte solution reservoir 12 and/or active agent molecules in the active agent solution reservoir 14. Undissociated electrolyte molecules and/or undissociated active agent molecules may thus be substantially prevented from transferring to the active agent solution reservoir 14 or the electrolyte solution reservoir 12 during storage of the device. As a result, changes to the active agent in the active agent solution reservoir 14, and/or decomposition of the active agent near the electrode 11 upon energization can be reduced or prevented.

The anion exchange membrane 13A and/or the cation exchange membrane 13C in the active electrode assemblies 10d, 10f, and 10h may also have a molecular weight cut-off, thus substantially blocking passage of electrolyte molecules in the electrolyte solution reservoir 12 and/or active agent molecules in the active agent solution reservoir 14.

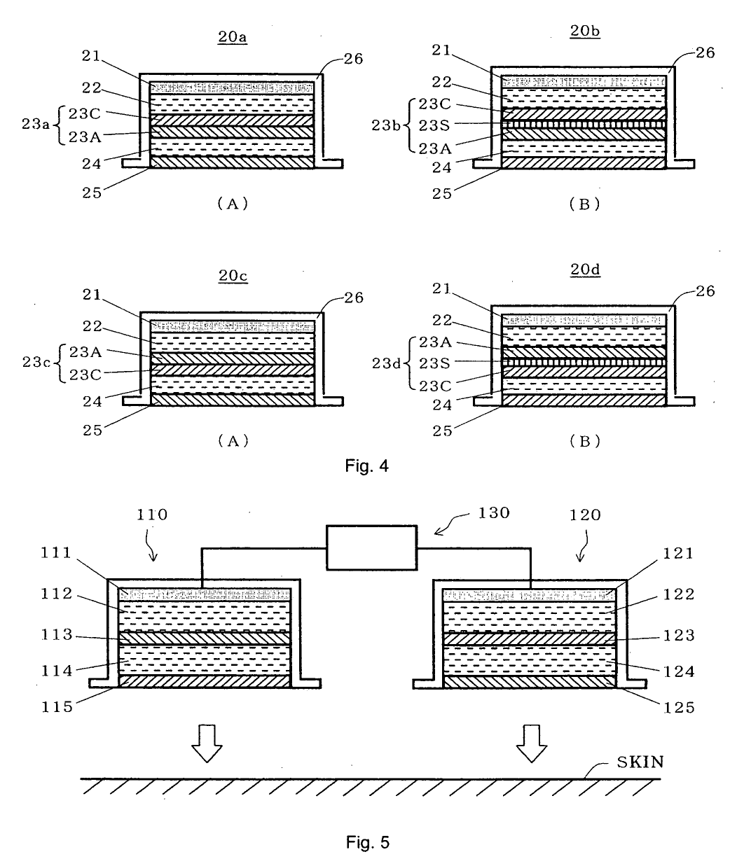

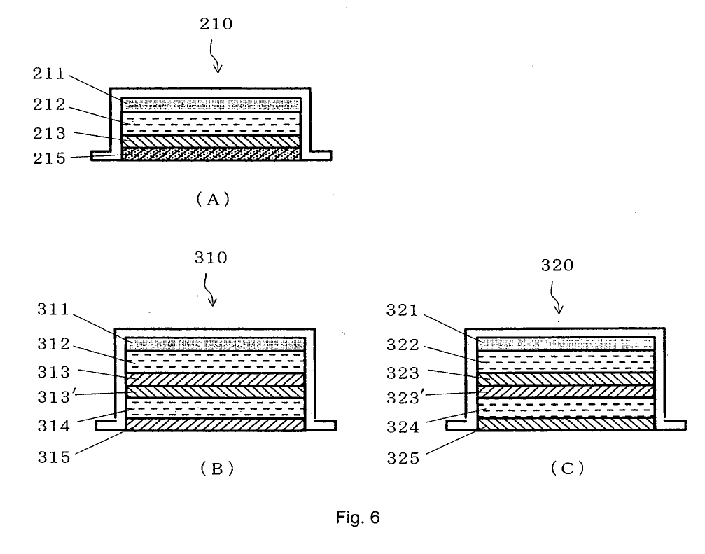

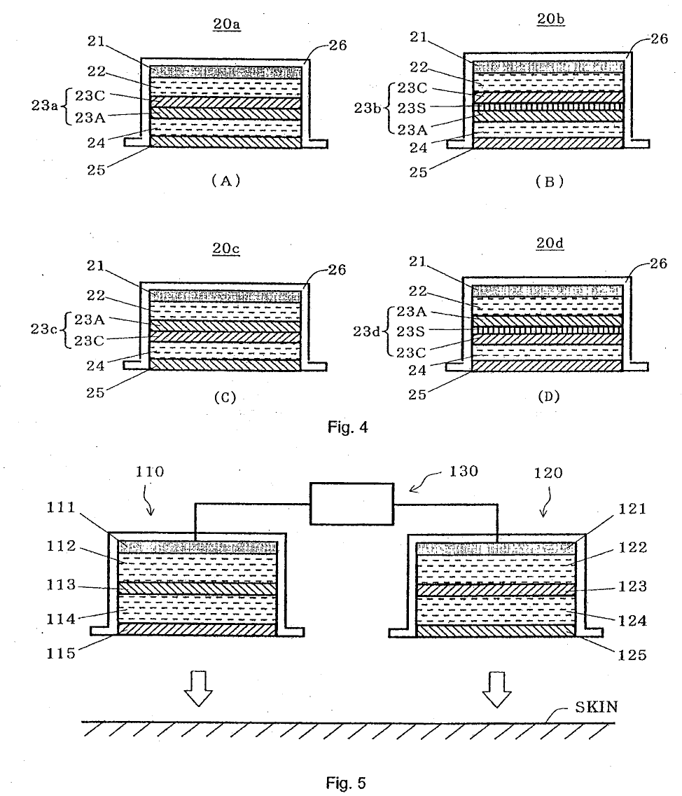

FIGS. 4A to 4D are explanatory sectional views showing the counter electrode assemblies 20a to 20d, each of which may be used as the counter electrode assembly 20 of the iontophoresis device X.

The counter electrode assembly 20a comprises: an electrode 21 connected to the supply line 32 of the electric power source 30; an electrolyte solution reservoir 22 that holds an electrolyte solution in contact with the electrode 21; a composite ion exchange membrane 23a arranged on the front surface side of the electrolyte solution reservoir 22 and having a composition similar to that of the composite ion exchange membrane 13e; an electrolyte solution reservoir 24 that holds an electrolyte solution, the electrolyte solution reservoir 24 being arranged on the front surface side of the composite ion exchange membrane 23a; and an anion exchange membrane 25 arranged on the front surface side of the electrolyte solution reservoir 24.

An electrolyte solution of a variety of compositions may be used for each of the electrolyte solution reservoirs 22 and 24. Using different electrolyte solutions in the electrolyte solution reservoirs 22 and 24 may provide desirable iontophoresis device performance. For example, an electrolyte solution that excels at preventing an electrode reaction at the electrode 21, or that excels in suppressing pH fluctuations in pH may be used in the electrolyte solution reservoir 22.

In addition, if the electrolyte solution reservoirs 22 and 24 hold different electrolyte solutions, arranging the composite ion exchange membrane 23a having two ion exchange membranes 23A and 23C opposite in polarity to each other between the electrolyte solution reservoir 22 and the electrolyte solution reservoir 24 may help to prevent mixing of the electrolyte solutions in the electrolyte solution reservoirs 22 and 24 during the storage of the device.

Furthermore, the composite ion exchange membrane 23a obtained by integrally coupling the anion exchange membrane 23A and the cation exchange membrane 23C may be used in the iontophoresis device X that includes the counter electrode assembly 20a. Construction of the counter electrode assembly 20a, automated production, and mass production may thus be simplified, and production costs may be reduced.

A counter electrode assembly 20b is similar the counter electrode assembly 20a except that it includes a composite ion exchange membrane 23b instead of the composite ion exchange membrane 23a. The composite ion exchange membrane 20b is similar to the composite ion exchange membrane 13f.

The counter electrode assembly 20b may make it more difficult for the electrolysis of water to occur between the anion exchange membrane 13A and the cation exchange membrane 13C because the anion exchange membrane 13A and the cation exchange membrane 13C are separated from each other by the semi-permeable membrane 13S.

The counter electrode assembly 20c is similar to the counter electrode assembly 20a except that the orientation of a composite ion exchange membrane 23c is opposite to that of the counter electrode assembly 20a. The counter electrode assembly 20d is similar to the counter electrode assembly 20b except that the orientation of a composite ion exchange membrane 23d is opposite to that of the counter electrode assembly 20d.

That is, in the counter electrode assemblies 20c and 20d, the anion exchange membrane 23A is arranged to contact the electrolyte solution of the electrolyte solution reservoir 22, and the cation exchange membrane 13C is arranged to contact with the electrolyte solution of the electrolyte solution reservoir 24.

The iontophoresis device X including the counter electrode assembly 20c and the iontophoresis device X including the counter electrode assembly 20d may make it more difficult for the electrolysis of water to occur between the anion exchange membrane 23A and the cation exchange membrane 23C because the semi-permeable membrane 13S separates the anion exchange membrane 13A and the cation exchange membrane 13C.

In the counter electrode assemblies 20a and 20c, the anion exchange membrane 23A and/or the cation exchange membrane 23C may have molecular weight cut-off that substantially blocks passage of electrolyte molecules in the electrolyte solution reservoir 22 and/or electrolyte molecules in the electrolyte solution reservoir 24. Undissociated electrolyte molecules may thus be prevented from transferring between the two electrolyte solution reservoirs 22 and 24 during the storage of the device. As a result, mixing of the electrolyte solutions in the electrolyte solution reservoirs 22 and 24 may be prevented.

In the counter electrode assemblies 20b and 20d, the anion exchange membrane 23A and/or the cation exchange membrane 23C may have a molecular weight cut-off that substantially blocks passage of electrolyte molecules in the electrolyte solution reservoir 22 and/or electrolyte molecules in the electrolyte solution reservoir 24. Mixing of the electrolyte solutions in the electrolyte reservoirs 22 and 24 may thus be prevented during storage.

In the iontophoresis device that includes an active electrode assembly and a counter electrode assembly corresponding to items (1) and (2) described below, members having the same composition can be used for the composite ion exchange membranes of both the electrode assemblies. This may greatly contribute to the simplification of production processes for the iontophoresis device X, make automated production and mass production easier, and reduce production costs.

In particular, an iontophoresis device that includes any one of items (3) to (6) described below may contribute to the simplification of production processes, automation of production, mass production, and a reduction in production costs because the orientation of the composite ion exchange membrane in the active electrode assembly is identical to that in the counter electrode assembly.

Furthermore, with an iontophoresis device that includes items (7) or (8) described below, the cation exchange membrane 15C in each of the composite ion exchange membranes 15a and 15b to be used in the active electrode assemblies 10a and 10b must be doped with active agent ions. However the same member may also be used in the composite ion exchange membranes of both electrode assemblies. This may greatly contribute to the simplification of production processes for the iontophoresis device, and may make automated production and mass production easier, and may reduce production costs.

(1) A combination of the active electrode assembly 10c, 10e, 10g, or 10i and the counter electrode assembly 20a or 20c

(2) A combination of the active electrode assembly 10d, 10f, 10h, or 10j and the counter electrode assembly 20b or 20d

(3) A combination of the active electrode assembly 10c or 10i and the counter electrode assembly 20c

(4) A combination of the active electrode assembly 10e or 10g and the counter electrode assembly 20a

(5) A combination of the active electrode assembly 10d or 10j and the counter electrode assembly 20d

(6) A combination of the active electrode assembly 10f or 10h and the counter electrode assembly 20b

(7) A combination of the active electrode assembly 10a and the counter electrode assembly 20a or 20c

(8) A combination of the active electrode assembly 10b and the counter electrode assembly 20b or 20d

The above description of illustrated embodiments, including what is described in the Abstract, is not intended to be exhaustive or to limit the embodiments to the precise forms disclosed. Although specific embodiments and examples are described herein for illustrative purposes, various equivalent modifications can be made without departing from the spirit and scope of the disclosure, as will be recognized by those skilled in the relevant art. The teachings provided herein of the various embodiments can be applied to other problem-solving systems devices, and methods, not necessarily the exemplary problem-solving systems devices, and methods generally described above.

For instance, the foregoing detailed description has set forth various embodiments of the systems, devices, and/or methods via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one embodiment, the present subject matter may be implemented via Application Specific Integrated Circuits (ASICs.) However, those skilled in the art will recognize that the embodiments disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

For example, an active agent may be administered through the following procedure. An active electrode assembly need not be provided with a counter electrode assembly. The active electrode assembly may be brought into contact with, for example, the biological interface of a subject, and an electric potential may applied to the active electrode assembly while a portion of the subject is brought into contact with a member to serve as ground.

Furthermore, although the active electrode assembly, the counter electrode assembly, and the power source are described as configured separately, it is also possible to incorporate the assemblies and power source in a single casing. In addition, an entire device incorporating the assemblies and power source may formed having a flat sheet or patch shape.

In addition, those skilled in the art will appreciate that the mechanisms of taught herein are capable of being distributed as a program product in a variety of forms, and that an illustrative embodiment applies equally regardless of the particular type of signal bearing media used to actually carry out the distribution. Examples of signal bearing media include, but are not limited to, the following: recordable type media such as floppy disks, hard disk drives, CD ROMs, digital tape, and computer memory; and transmission type media such as digital and analog communication links using TDM or IP based communication links (e.g., packet links.)

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet including but not limited to U.S. Provisional Patent Application Ser. No. 60/718,019, filed Sep. 15,2005, and Japan Patent Application No. 2005-238026, filed Aug. 18, 2005, are incorporated herein by reference, in their entirety.

Aspects of the embodiments can be modified, if necessary, to employ systems, circuits, and concepts of the various patents, applications, and publications to provide yet further embodiments.

These and other changes can be made to the embodiments in light of the above-detailed description. In general, in the following claims, the terms used should not be construed to limit the invention to the specific embodiments disclosed in the specification and the claims, but should be construed to include all possible embodiments along with the full scope of equivalents to which such claims are entitled. Accordingly, the scope of the invention shall only be construed and defined by the scope of the appended claims.

What is claimed is:

1. An iontophoresis device, comprising an electrode assembly that includes a composite ion exchange membrane, the composite ion exchange membrane comprising a first ion exchange membrane of a first polarity and a second ion exchange membrane of a second polarity placed on and integrally coupled to the first ion exchange membrane.

2. The iontophoresis device according to claim 1 wherein:
the electrode assembly further comprises a first electrode, and a first electrolyte solution reservoir that holds an electrolyte solution that contacts the first electrode;
the composite ion exchange membrane is placed on a front surface side of the first electrolyte solution reservoir;
the first ion exchange membrane is placed on a front surface side of the second ion exchange membrane; and
the first ion exchange membrane is doped with active agent ions of the first polarity.

3. The iontophoresis device according to claim 1 wherein:
the electrode assembly further comprises a first electrode, a first electrolyte solution reservoir that holds an electrolyte solution that contacts with the first electrode, and an active agent solution reservoir that holds an active agent solution containing active agent ions of the first polarity, the active agent solution reservoir being placed on a front surface side of the first electrolyte solution reservoir; and
the composite ion exchange membrane is placed between the first electrolyte solution reservoir and the active agent solution reservoir.

4. An iontophoresis device, comprising an electrode assembly that includes a composite ion exchange membrane, the composite ion exchange membrane comprising a first ion exchange membrane of a first polarity, a semi-permeable membrane placed on and integrally coupled to the first ion exchange membrane, and a second ion exchange membrane of a second polarity placed on and integrally coupled to the semi-permeable membrane.

5. The iontophoresis device according to claim 4 wherein:
the electrode assembly further comprises a first electrode, and a first electrolyte solution reservoir that holds an electrolyte solution that contacts the first electrode;
the composite ion exchange membrane is placed on a front surface side of the first electrolyte solution reservoir;
the first ion exchange membrane is placed on a front surface side of the second ion exchange membrane; and
the first ion exchange membrane is doped with active agent ions of the first polarity.

6. The iontophoresis device according to claim 4 wherein:
the electrode assembly further comprises a first electrode, a first electrolyte solution reservoir that holds an electrolyte solution that contacts with the first electrode, and an active agent solution reservoir that holds an active agent solution containing active agent ions of the first polarity, the active agent solution reservoir being placed on a front surface side of the first electrolyte solution reservoir; and
the composite ion exchange membrane is placed between the first electrolyte solution reservoir and the active agent solution reservoir.

7. An iontophoresis device, comprising:
an active electrode assembly holding active agent ions of a first polarity; and
a counter electrode assembly as a counter electrode of the active electrode assembly, the counter electrode assembly comprising:
a counter electrode;
a first counter electrolyte solution reservoir that holds an electrolyte solution in contact with the counter electrode;
a second counter electrolyte solution reservoir that holds an electrolyte solution, the second counter electrolyte solution reservoir being placed on a front surface side of the first counter electrolyte solution reservoir; and
a composite ion exchange membrane placed between the first counter electrolyte solution reservoir and the second counter electrolyte solution reservoir, the composite ion exchange membrane including a first ion exchange membrane of a first polarity and a second ion exchange membrane of a second polarity stacked on and integrally coupled to the first ion exchange membrane.

8. An iontophoresis device, comprising:
an active electrode assembly holding active agent ions of a first polarity; and
a counter electrode assembly as a counter electrode of the active electrode assembly, the counter electrode assembly comprising:
a counter electrode;
a first counter electrolyte solution reservoir that holds an electrolyte solution in contact with the counter electrode;
a second counter electrolyte solution reservoir that holds an electrolyte solution, the second counter electrolyte solution reservoir being placed on a front surface side of the first counter electrolyte solution reservoir; and
a composite ion exchange membrane placed between the first counter electrolyte solution reservoir and the second counter electrolyte solution reservoir, the composite ion exchange membrane including a first ion exchange membrane of a first polarity, a semi-permeable membrane integrally coupled to the first ion exchange membrane, and a second ion exchange membrane of a second polarity integrally coupled to the semi-permeable membrane.

9. A composite ion exchange membrane for iontophoresis, comprising:
a first ion exchange membrane of a first polarity; and
a second ion exchange membrane of a second polarity integrally coupled to the first ion exchange membrane.

10. A composite ion exchange membrane for iontophoresis, comprising:
a first ion exchange membrane of a first polarity;
a semi-permeable membrane integrally coupled to the first ion exchange membrane; and
a second ion exchange membrane of a second polarity integrally coupled to the semi-permeable membrane.
